(19) United States
(12) Patent Application Publication
(10) Pub. No.: US 2023/0298201 A1
(43) Pub. Date: Sep. 21, 2023

(54) ELECTRONIC DEVICE, METHOD, AND COMPUTER READABLE RECORDING MEDIUM FOR OBTAINING POSTURE INFORMATION INDICATING POSTURE OF BODY BASED ON MARKER ATTACHED TO BODY PART

(71) Applicant: NCSOFT CORPORATION, Seongnam-si (KR)

(72) Inventors: Sungbum Park, Seongnam-si (KR); Sangjun An, Seongnam-si (KR)

(73) Assignee: NCSOFT CORPORATION, Seongnam-si (KR)

(21) Appl. No.: 18/182,553

(22) Filed: Mar. 13, 2023

(30) Foreign Application Priority Data
Mar. 15, 2022 (KR) 10-2022-003285

(51) Int. Cl. G06T 7/70; G06V 40/10; G06T 7/11

(52) U.S. Cl. CPC G06T 7/70; G06T 7/11; G06V 40/10; G06T 2207/30196; G06T 2207/30204

(57) ABSTRACT

An electronic device according to an embodiment includes a memory for storing instructions, and at least one processor, wherein when the instructions are executed, the at least one processor is configured to identify a video capturing a body and one or more markers attached to the body, obtain a first information including an angle and a position of at least one first joint among joints being included in the body, based on the one or more markers in the video, obtain a second information including a position of at least one second joint among the joints being included in the body, based on at least part of the video in which the body is captured among the body and the one or more markers in the video, and obtain a third information indicating a posture of the body based on the interconnection of the at least one first joint and the at least one second joint in the video, based on the first information and the second information.

### CROSS-REFERENCE TO RELATED APPLICATIONS

This application is based on and claims priority under 35 U.S.C 119 to Korean Patent Application No. 10-2022-0032385 filed on, Mar. 15, 2022, in the Korean Intellectual Property Office, the disclosures of which is incorporated by reference herein their entirety.

### BACKGROUND

### Field

Various embodiments relate to an electronic device, method, and recording medium for obtaining posture information indicating a posture of a body based on a marker attached to a body part.

### Description of Related Art

Recently, interest in a technique for expressing a posture of a body within a virtual two-dimensional space or a virtual three-dimensional space is increasing by capturing the body and interpreting the captured video through a neural network. The neural network may mean a model with the ability capable of solving a specific problem by adjusting the strength of synaptic coupling through learning about a node that has formed a network through the synaptic coupling. For example, the posture of the body may be reconstructed within a virtual space by obtaining a plurality of points corresponding to a body part through the neural network and connecting the obtained plurality of points to each other, based on the inputted image or video.

### SUMMARY

In order to reconstruct a posture of a body in a virtual space, an electronic device may obtain points corresponding to a plurality of body parts forming the body. For example, the electronic device may obtain points corresponding to a distal part of the body, such as a wrist and an ankle of the body, or another body part spaced apart from the distal part of the body, such as a hip joint and a shoulder. Since the recognition rate of the distal part of the body is relatively lower than that of another body part spaced apart from the distal part, the electronic device may not accurately identify the point corresponding to the distal part of the body. In case that a motion sensor or a depth sensor is used when capturing a video to accurately identify the distal part, a problem that an error occurs due to the sensor, or constraints to be considered by a photographer when capturing the video increases may occur.

The electronic device, method, and recording medium according to various embodiments may obtain posture information indicating the posture of the body based on a marker attached to the body part.

The technical problems to be achieved in this document are not limited to those described above, and other technical problems not mentioned herein will be clearly understood by those having ordinary knowledge in the art to which the present disclosure belongs, from the following description.

An electronic device according to an embodiment may comprise a memory for storing instructions, and at least one processor, wherein when the instructions are executed, the at least one processor may be configured to identify a video capturing a body and one or more markers attached to the body, obtain first information including an angle and a position of at least one first joint among joints being included in the body, based on the one or more markers in the video, obtain a second information including a position of at least one second joint among the joints being included in the body, based on at least part of the video in which the body is captured among the body and the one or more markers in the video, and obtain third information indicating a posture of the body based on the interconnection of the at least one first joint and the at least one second joint in the video, based on the first information and the second information.

According to an embodiment, an operating method of an electronic device may comprise identifying a video capturing a body and one or more markers attached to the body, obtaining first information including an angle and a position of at least one first joint among joints being included in the body, based on the one or more markers in the video, obtaining a second information including a position of at least one second joint among the joints being included in the body, based on at least part of the video in which the body is captured among the body and the one or more markers in the video, and obtaining third information indicating a posture of the body based on the interconnection of the at least one first joint and the at least one second joint in the video, based on the first information and the second information.

According to an embodiment, a computer readable storage medium storing one or more programs, when executed by at least one processor of electronic device, the one or more program causes electronic device to obtain a first region capturing a marker attached to a body and second region capturing the body and at least partially overlapped to the first region, from a video capturing the body, identify a position of a designated body part among a plurality of body parts of the body, based on the marker being included in the first region, identify a position of the plurality of body parts being included in the body in the second region, from the second region, obtaining an information indicating a posture of the body, based on the position of the plurality of body parts being identified in the second region and the position of the designated body part being identified based on the marker.

An electronic device, method, and recording medium according to an embodiment can identify the position of some of the joints included in a body based on a marker, so that a posture of the body can be accurately reconstructed within a virtual space without a separate motion sensor or depth sensor. Since the electronic device, method, and recording medium according to an embodiment can reconstruct the posture of the body based on the marker, it can reduce an error due to the sensor, and constraints that a videographer should consider.

The effects that can be obtained from the present disclosure are not limited to those described above, and any other effects not mentioned herein will be clearly understood by those having ordinary knowledge in the art to which the present disclosure belongs, from the following description.

### BRIEF DESCRIPTION OF THE DRAWINGS

The above and/or other aspects of the disclosure will be more apparent by describing certain embodiments of the disclosure with reference to the accompanying drawings, in which:

FIG. 1 is a simplified block diagram illustrating a functional configuration of an electronic device according to an embodiment;

FIG. 2 is an exemplary diagram for describing a neural network obtained by an electronic device according to an embodiment from a set of parameters stored in a memory;

FIG. 3 illustrates an example of an environment including an electronic device according to an embodiment;

FIG. 4 illustrates an example of a method of identifying regions and a marker in a video by an electronic device according to an embodiment;

FIG. 5 illustrates an example of a method of identifying at least one first joint of a body based on a marker by an electronic device according to an embodiment;

FIG. 6 illustrates an example of a method of identifying at least one second joint of a body based on a video by an electronic device according to an embodiment;

FIG. 7 illustrates an example of information indicating a posture of a body obtained by an electronic device according to an embodiment;

FIG. 8 is a flowchart for illustrating an operation of an electronic device according to an embodiment;

### DETAILED DESCRIPTION

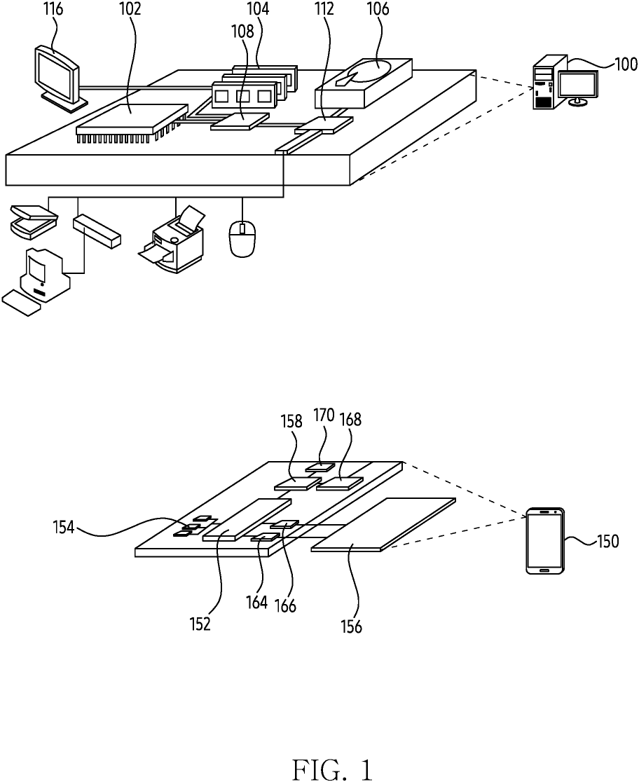

FIG. 1 is a simplified block diagram illustrating a functional configuration of an electronic device according to an embodiment.

Referring to FIG. 1, an electronic device 100 according to an embodiment may include a processor 102, a memory 104, a storage device 106, a high-speed controller 108 (e.g., northbridge, MCH (main controller hub)), a low-speed controller 112 (e.g., southbridge, ICH (I/O (input/output) controller hub)). Within the electronic device 100, each of the processor 102, the memory 104, the storage device 106, the high-speed controller 108, and the low-speed controller 112 may be interconnected by using various buses. For example, the processor 102 may process instructions for execution within the electronic device 100 to display graphic information for a GUI (graphical user interface) on an external input/output device such as a display 116 connected to the high-speed controller 108. The instructions may be included in the memory 104 or the storage device 106. The instructions, when executed by the processor 102, may cause the electronic device 100 to perform one or more operations described above and/or one or more operations to be described below. According to embodiments, the processor 102 may be configured with a plurality of processors including a communications processor and a GPU (graphical processing unit.

For example, the memory 104 may store information in the electronic device 100. For example, the memory 104 may be a volatile memory unit or units. For another example, the memory 104 may be a non-volatile memory unit or units. For still another example, the memory 104 may be another type of computer-readable medium, such as a magnetic or optical disk.

For example, the storage device 106 may provide the electronic device 100 with a mass storage space. For example, the storage device 106 may be a computer-readable medium, such as a hard disk device, an optical disk device, a flash memory, a solid-state memory device, or an array of devices in a SAN (storage area network).

For example, the high-speed controller 108 may manage bandwidth-intensive operations for the electronic device 100, while the low-speed controller 112 may manage low bandwidth-intensive operations for the electronic device 100. For example, the high-speed controller 108 may be coupled with the memory 104 and may be coupled with the display 116 through the GPU or accelerator, while the low-speed controller 112 may be coupled with the storage device 106 and may be coupled with various communication ports (e.g., USB (universal serial bus), Bluetooth, ethernet, wireless ethernet) for communication with an external electronic device (e.g., a keyboard, a transducer, a scanner), or a network device (e.g., a switch or a router).

According to an embodiment, an electronic device 150 may be another example of the electronic device 100. The electronic device 150 may include an input/output device such as a processor 152, a memory 154, a display 156 (e.g., an OLED (organic light emitting diode) display or another suitable display), a communication interface 158, and a transceiver 162. Each of the processor 152, the memory 154, the input/output device, the communication interface 158, and the transceiver 162 may be interconnected by using various buses.

For example, the processor 152 may process instructions included in the memory 154 to display graphical information for the GUI on the input/output device. The instructions, when executed by the processor 152, may cause the electronic device 150 to perform one or more operations described above and/or one or more operations to be described below. For example, the processor 152 may interact with a user through a display interface 164 and a control interface 166 coupled to the display 156. For example, the display interface 164 may include a circuit for driving the display 156 to provide visual information to the user, and the control interface 166 may include a circuit for receiving commands received from the user and converting the commands to provide them to the processor 152. According to embodiments, the processor 152 may be implemented as a chipset of chips including analog and digital processors.

For example, the memory 154 may store information in the electronic device 150. For example, the memory 154 may include at least one of one or more volatile memory units, one or more non-volatile memory units, or the computer-readable medium.

For example, the communication interface 158 may perform wireless communication between the electronic device 150 and the external electronic device through various communication techniques such as a cellular communication technique, a Wi-Fi communication technique, an NFC technique, or a Bluetooth communication technique, based on the interworking with the processor 152. For example, the communication interface 158 may be coupled to the transceiver 168 to perform the wireless communication. For example, the communication interface 158 may be further coupled with a GNSS (global navigation satellite system) reception module 170 to obtain position information of the electronic device 150.

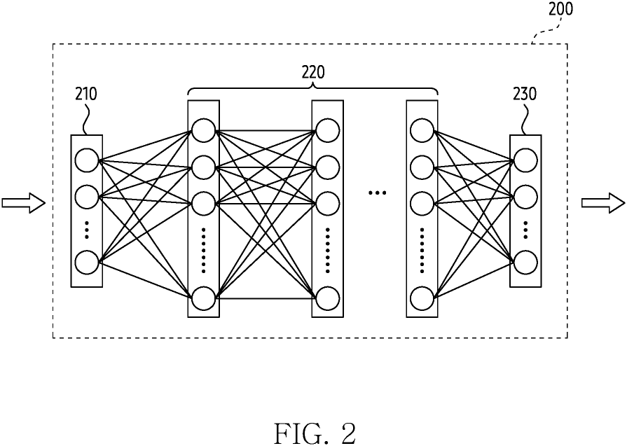

FIG. 2 is an exemplary diagram for describing a neural network obtained by an electronic device according to an embodiment from a set of parameters stored in a memory.

Referring to FIG. 2, the set of parameters related to a neural network 200 may be stored in a memory (e.g., a memory 104 of FIG. 1) of an electronic device (e.g., an electronic device 100 of FIG. 1) according to an embodiment. The neural network 200 is a recognition model implemented with software or hardware that mimics the computational power of a biological system by using a large number of artificial neurons (or nodes). The neural network 200 may perform a human cognitive action or a learning process through artificial neurons. The parameters related to the neural network 200 may indicate, for example, weights assigned to a plurality of nodes included in the neural network 200 and/or connections between the plurality of nodes. The number of the neural networks 200 stored in the memory 104 is not limited as illustrated in FIG. 2, and the set of parameters corresponding to each of a plurality of neural networks may be stored in the memory 104.

The model trained by the electronic device 100 according to an embodiment may be implemented based on the neural network 200 indicated based on the set of a plurality of parameters stored in the memory 104. The neurons of the neural network 200 corresponding to the model may be classified along a plurality of layers. The neurons may be indicated by a connection line connecting between a specific node included in a specific layer and another node included in a different another layer from the specific layer, and/or a weight assigned to the connection line. For example, the neural network 200 may include an input layer 210, hidden layers 220, and an output layer 230. The number of the hidden layers 220 may vary according to an embodiment.

The input layer 210 may receive a vector indicating input data (e.g., a vector having elements corresponding to the number of nodes included in the input layer 210). Based on the input data, signals generated at each of the nodes in the input layer 210 may be transmitted from the input layer 210 to the hidden layers 220. The output layer 230 may generate output data of the neural network 200 based on one or more signals received from the hidden layers 220. The output data may include, for example, the vector having elements mapped to each of the nodes included in the output layer 230.

The hidden layers 220 may be positioned between the input layer 210 and the output layer 230 and may change the input data transmitted through the input layer 210. For example, as the input data received through the input layer 210 propagates sequentially from the input layer 210 along the hidden layers 220, the input data may be gradually changed based on the weight connecting nodes of different layers.

As described above, each of the layers (e.g., the input layer 210, the hidden layers 220, and the output layer 230) included in the neural network 200 may include the plurality of nodes. The hidden layers 220 may be a convolution filter or a fully connected layer in a CNN (convolutional neural network), or may be a various type of a filter or a layer bound based on a special function or feature.

A structure in which the nodes are connected between the different layers is not limited to an example of FIG. 2. In an embodiment, the one or more hidden layers 220 may be a layer based on a recurrent neural network (RNN) in which an output value is re-entered to the hidden layer of the current time. In an embodiment, based on a LSTM (Long Short-Term Memory), the neural network 200 may further include one or more gates (and/or filters) for discarding at least one of the values of nodes, maintaining them for a relatively long period of time, or maintaining them for a relatively short period of time. The neural network 200 according to an embodiment may include the numerous hidden layers 220 to form a deep neural network. Training the deep neural network is called deep learning. The node included in the hidden layers 220 may be referred to as a hidden node.

The nodes included in the input layer 210 and the hidden layers 220 may be connected to each other through a connection line having the weights, and the nodes included in the hidden layers 220 and the output layer 230 may also be connected to each other through the connection line having the weights. Tuning and/or training the neural network 200 may mean changing the weights between the nodes included in each of the layers (e.g., the input layer 210, the hidden layers 220, and/or the output layer 230) included in the neural network 200. The tuning of the neural network 200 may be performed, for example, based on supervised learning and/or unsupervised learning.

The electronic device 100 according to an embodiment may train a model 240 based on the supervised learning. The supervised learning may mean training the neural network 200 by using a set of paired input data and output data. For example, the neural network 200 may be tuned to reduce the difference between the output data output from the output layer 230 and the output data included in the set while receiving the input data included in the set. As the number of sets increases, the neural network 200 may generate the output data generalized by the one or more sets with respect to other input data distinct from the set.

The electronic device 100 according to an embodiment may tune the neural network 200 based on reinforcement learning in the unsupervised learning. For example, the electronic device 100 may change policy information used by the neural network 200 to control an agent based on interaction between the agent and an environment. The electronic device 100 according to an embodiment may cause the change in the policy information by the neural network 200 to maximize the target and/or compensation of the agent by the interaction.

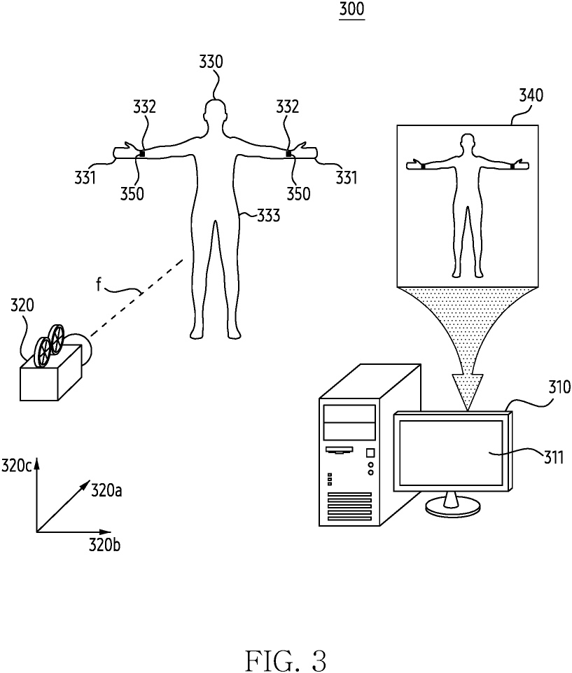

FIG. 3 illustrates an example of an environment including an electronic device according to an embodiment.

Referring to FIG. 3, an environment 300 according to an embodiment may include an electronic device 310 and one or more cameras 320. Since the electronic device 310 of FIG. 3 may be substantially the same as an electronic device 100 and/or an electronic device 150 of FIG. 1, an overlapping description will be omitted.

According to an embodiment, the electronic device 310 may be a terminal or a server owned by a user. For example, the electronic device 310 may include at least one of a personal computer (PC) such as a laptop and a desktop, a smartphone, a smartpad, and a tablet PC (Personal Computer).

According to an embodiment, the electronic device 310 may include a memory for storing instructions, at least one processor, and a display 311. The memory of the electronic device 310 may be substantially the same as a memory 104 or a memory 154 of FIG. 1, at least one processor of the electronic device 310 may be substantially the same as a processor 102 or a processor 152 of FIG. 1, and the display 311 of the electronic device 310 may be substantially the same as a display 116 or a display 156 of FIG. 1, so an overlapping description will be omitted.

The one or more cameras 320 may be used to capture a body 330. The camera 320 may be, for example, at least one of a digital camera, an action camera, and a gimbal camera, for capturing the body 330, but is not limited thereto. In FIG. 3, the camera 320 and the electronic device 310 are illustrated as separate devices, but this is for convenience of description. According to embodiments, the camera 320 may be a partial component of the electronic device 310 included in the electronic device 310.

According to an embodiment, the camera 320 may move relatively with respect to the body 330. For example, the camera 320 may rotate with respect to the body 330. The camera 320 may rotate based on at least one of a roll axis 320a, a pitch axis 320b, and a yaw axis 320c of the camera 320. The roll axis 320a of the camera 320 may mean a virtual line extending along a direction parallel to an optical axis f of the camera 320. The roll axis 320a of the camera 320 may extend along the front or rear of the camera 320. The pitch axis 320b of the camera 320 may mean the virtual line extending along a direction perpendicular to the roll axis 320a of the camera 320. For example, the pitch axis 320b of the camera 320 may extend along the left side or the right side of the camera 320 based on the camera 320. The yaw axis 320c of the camera 320 may mean the virtual line extending along a direction perpendicular to both the roll axis 320a of the camera 320 and the pitch axis 320b of the camera 320. For example, the yaw axis 320c of the camera 320 may extend along upward or downward of the camera 320 based on the camera 320. In case that the camera 320 is disposed on a ground, the yaw axis 320c of the camera 320 may be perpendicular with respect to the ground. According to an embodiment, the camera 320 may move linearly with respect to the body 330. For example, the camera 320 may move in a direction away from the body 330 or in a direction becoming closer to the body 330 along at least one of the roll axis 320a, the pitch axis 320b, and the yaw axis 320c of the camera 320.

According to an embodiment, the body 330 may take a specific posture while being captured by the camera 320. The camera 320 may obtain a video 340 by capturing the body 330 which takes the specific posture. For example, the video 340 may include an image obtained by capturing the body 330 which takes the specific posture. The image may be referred to a still image. For another example, the video 340 may include a video obtained by capturing the body 330 which takes the specific posture, for a designated period of time.

According to an embodiment, a marker 350 may include a pattern in the form of two-dimension so as to be easily identified by the electronic device 310. For example, the marker 350 may include a pattern made of black and white. The marker 350 may include, for example, a QR code (quick response code), but is not limited thereto. According to embodiments, the marker 350 may include a pattern in the form of a bar code. For another example, the marker 350 may include a pattern formed of a combination of saturation, light and shade, or color, that is contrasted in a color space. According to an embodiment, the shape of the marker 350 may be a two-dimensional polygon (e.g., a triangle and/or a quadrangle), but is not limited thereto.

According to an embodiment, the marker 350 may be attached to the body 330. The marker 350 may be, for example, detachably attached to the body 330 or drawn on the body 330. According to an embodiment, the marker 350 may be attached to a distal part 331 of the body 330 among body parts included in the body 330. According to an embodiment, the marker 350 may include a plurality of markers 350 disposed in each of different designated body parts included in the body 330. For example, the marker 350 may be disposed on a first designated body part of the body 330 and a second designated body part of the body 330 spaced apart from the first body part. The first designated body part and the second designated body part may be, for example, a left wrist and a right wrist of the body 330, respectively, but are not limited thereto. According to an embodiment, the marker 350 may be in contact with at least a part of the distal part 331 of the body 330. For example, the marker 350 may be in contact with a part of the distal part 331 so as to correspond to at least one first joint 332 included in the distal part 331. For another example, the marker 350 may be in contact with the distal part 331 while surrounding the entire distal part 331. In FIG. 1, an example in which the distal part 331 includes the left wrist and/or the right wrist of the body 330 is illustrated, but embodiments are not limited thereto. For example, the distal part 331 may include a left ankle and/or a right ankle.

According to an embodiment, the number of the plurality of markers 350 may correspond to the number of different body parts of the body 330. For example, the number of the plurality of markers 350 may be two, and one plurality of markers 350 may be disposed in the first designated body part (e.g., the right wrist) and the second designated body part (e.g., the left wrist) of the body 330, respectively. According to an embodiment, the number of the plurality of markers 350 may not correspond to the number of different body parts of the body 330. For example, the plurality of markers 350 may be disposed on one designated body part among the different designated body parts.

According to an embodiment, the shapes of the plurality of markers 350 disposed on the different designated body parts may be the same. For example, the shape of the marker 350 disposed on the first designated body part may be substantially the same as the shape of the marker 350 disposed on the second designated body part.

According to an embodiment, the shape of the plurality of markers 350 disposed on one designated body part among the different designated body parts may be different from each other. For example, among a plurality of markers 350 disposed on the right wrist, the shape of the marker 350 disposed on one surface of the right wrist may be different from the shape of the marker 350 disposed on the other surface of the right wrist.

According to an embodiment, the one or more cameras 320 may transmit the body 330 and the video 340 capturing one or more markers 350 attached to the body 330 to the electronic device 310. The camera 320 may be connected to the electronic device 310 by wire or wirelessly and may transmit the captured video 340 to the electronic device 310. For example, the video 340 may be the image or the video in the form of two-dimension. According to an embodiment, the electronic device 310 may display the received video 340 on the display 311 based on receiving the video 340.

According to an embodiment, the processor of the electronic device 310 may identify the body 330 and the video 340 in which the one or more markers 350 attached to the body are captured. For example, the processor of the electronic device 310 may identify the first region in the video including the one or more markers 350 and the distal part 331 of the body 330 corresponding to at least one first joint 332, and the second region in the video 340 distinguished by the body 330, based on a first model which received the video 340. According to an embodiment, the first model may include, for example, a neural network (e.g., a neural network 200 of FIG. 2) pre-learned to identify the body 330 or the designated body parts included in the body 330 in the video 340. For example, the first model may include a convolution neural network, but is not limited thereto.

According to an embodiment, the processor of the electronic device 310 may obtain first information including the position and angle of the at least one first joint 332 among joints included in the body 330, based on the one or more markers 350, in the video 340. The at least one first joint 332 may include at least one joint of the body 330 included in the distal part 331 of the body 330 among the joints included in the body 330. For example, the at least one first joint 332 may include at least one of a left wrist joint, a right wrist joint, a left ankle joint, or a right ankle joint, but is not limited thereto.

According to an embodiment, the processor of the electronic device 310 may obtain the first information based on identifying the marker 350 in the video 340. For example, in case that the marker 350 includes an outline and/or a patter based on a two-dimensional figure, the processor may identify the marker 350 based on an algorithm that recognizes the outline. For example, in case that the marker 350 includes the pattern in the form of a QR code, the processor may identify the marker 350 through the algorithm used to recognize the QR code. According to an embodiment, the processor may obtain a plurality of coordinates indicating different points of the marker 350 in the video 340. The processor may obtain the first information based on comparing each of the one or more markers 350 captured in video 340 and the one or more markers 350 captured in another video pre-stored in the memory. For example, before the video 340 is obtained, the processor may pre-store another video (e.g., the image or the video) in which the marker 350 is captured in the memory. In case that there are a plurality of markers 350, the processor may pre-store a plurality of different videos which are obtained by capturing each of the plurality of markers 350, in the memory. Before the video 340 is obtained, the processor may pre-store coordinates of a plurality of points included in the marker 350 in another video, in the memory. The processor may identify the position and angle of the marker 350 in the video 340 by comparing the plurality of coordinates obtained from the marker 350 in the video 340 with the plurality of coordinates obtained from the marker 350 in another video. Based on identifying the position and angle of the marker 350 in video 340, the processor may obtain the first information on the position and angle of the at least one first joint 332 to which the marker 350 is attached.

For example, in case that the marker 350 is not attached to the distal part 331 of the body 330, the distal part 331 of the body 330 may have a relatively small recognition rate by the neural network compared to other body parts of the body 330. As the recognition rate is small, in case that the marker 350 is not attached to the distal part 331 of the body 330, the electronic device 310 may not be able to accurately obtain information on the position and angle of the at least one first joint 332 included in the distal part 331. In order to increase the recognition rate, in case that a motion sensor is attached to the body 330, a problem in which an error due to the motion sensor increases and a problem in which the user without specialized knowledge does not smoothly proceed with capturing may occur. In order to increase the recognition rate, in case that the camera 320 capable of performing a depth sensor function is used, a problem in that the amount of calculation of the electronic device 310 increases may occur. The electronic device 310 according to an embodiment may identify the first information on the position and angle of at least one first joint 332 based on the marker 350, so that the position and angle of at least one first joint 332 may be obtained relatively easily. The electronic device 310 according to an embodiment may obtain the first information based on the marker 350, thereby reducing the amount of calculation of the electronic device 310.

According to an embodiment, the processor of the electronic device 310 may obtain second information including the position of at least one second joint 333 among joints included in the body 330 based on at least a part of the video 340 in which the body 330 is captured, among the body 330 and the one or more markers 350, in the video 340. The at least one second joint 333 may include joints included in the body 330 except for the at least one first joint 332 included in the distal part 331 of the body 330. For example, at least one second joint 333 may include at least one of a hip joint, a spine, a shoulder joint, an elbow joint, and a knee joint of the body 330, but is not limited thereto.

According to an embodiment, the processor of the electronic device 310 may obtain the second information based on identifying the second region in the video 340 distinguished by the body 330. Based on the second model which received the second region, the processor may obtain the second information indicating a probability that at least one of the at least one first joint and the at least one second joint 333 exists in the virtual two-dimensional space. For example, the second information may indicate the probability that the at least one first joint 332 and the at least one second joint 333 exist in the form of a heat map. According to an embodiment, the second model may include the neural network (e.g., the neural network 200 of FIG. 2) pre-learned to identify the position of joints included in the body 330. For example, the second model may include a convolution neural network, but is not limited thereto.

According to an embodiment, the processor of the electronic device 310 may obtain third information indicating the posture of the body 330 based on the interconnection of the at least one first joint 332 and the at least one second joint 333 in the video 340 based on the first information and the second information.

As described above, the electronic device 310 according to an embodiment may identify the position of the at least one first joint 332 included in the distal part 331 of the body 330 having a relatively low recognition rate based on the marker 350. Since the electronic device 310 according to an embodiment may recognize the distal part 331 of the body 330 based on the marker 350, it is possible to quickly reconstruct the posture of the body 330 without the motion sensor or the depth sensor.

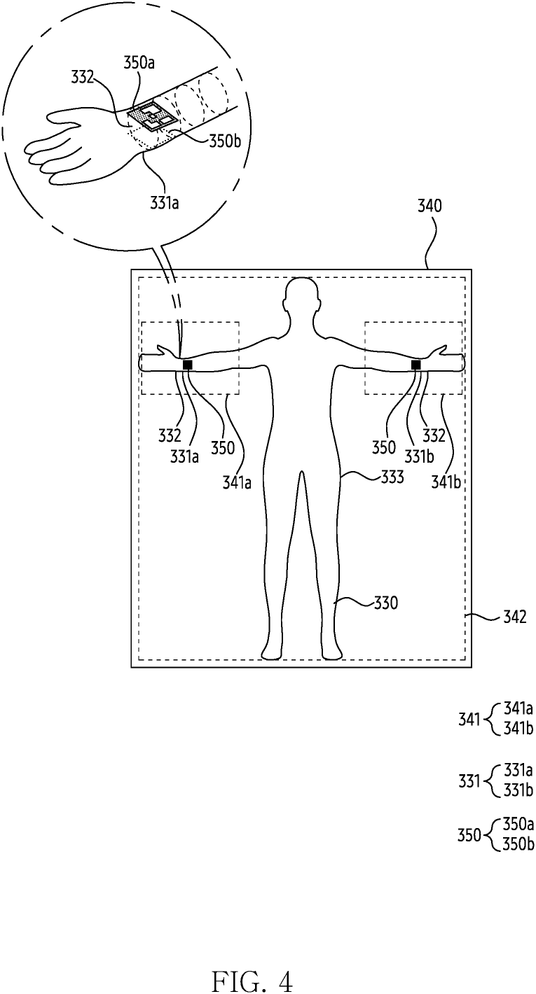

FIG. 4 illustrates an example of a method of identifying regions and a marker in a video by an electronic device according to an embodiment.

Referring to FIG. 4, according to an embodiment, the processor of the electronic device (e.g., an electronic device 310 of FIG. 3) may identify a first region 341 and a second region 342 in a video 340 based on a first model (e.g., a neural network 200 of FIG. 2) which is received the video 340. The first region 341 may mean a part of the video 340 including one or more markers 350 and a distal part 331 corresponding to at least one first joint 332. For example, the first region 341 may include a 1-1 region 341a including a first designated body part 331a, which is the distal part 331 of the body 330, and a 1-2 region 341b including a second designated body part 331b in the video 340. The second region 342 may mean at least a part of the video 340 distinguished by the body 330. For example, the second region 342 may partially overlap the first region 341 by including at least a part of the video 340 partitioned, in the body 330. For another example, the second region 342 may have a size corresponding to the video 340 by including the entire region of the video 340.

According to an embodiment, a plurality of markers 350 may be disposed on the first joints 332 included in different distal parts 331 of the body 330. For example, the plurality of markers 350 may be disposed on the first designated body part 331a and the second designated body part 331b of the body 330. According to an embodiment, a shape of the plurality of markers 350 attached to different designated body parts 331a and 331b of the body 330 may be the same each other. For example, the shape of the marker 350 disposed on the first designated body part 331a of the body 330 may be substantially the same as the shape of the marker 350 disposed on the second designated body part 331b of the body 330 corresponding to the first designated body part 331a. For another example, the shape of the marker 350 disposed on the first designated body part 331a of the body 330 may be substantially the same as the shape of the marker 350 disposed on the second designated body part 331b of the body 330 symmetrical to the first designated body part 331a. Since the different designated body parts 331a and 331b of the body 330 are distinguished through the first model, the electronic device 310 according to an embodiment may identify the first joints 332 even in case that the shape of the marker 350 disposed on the different designated body parts 331a and 331b are the same.

According to an embodiment, among the different designated body parts 331a and 331b, the shapes of the plurality of markers 350 disposed on one designated body part 331a may be different from each other. For example, the plurality of markers 350 may include a first marker 350a and a second marker 350b attached to the first designated body part 331a. The first marker 350a may be disposed on one surface of the first designated body part 331a, and the second marker 350b may have a shape distinct from the first marker 350a and may be disposed on the other surface of the first designated body part 331a facing one surface of the first designated body part 331a. For example, one surface of the first designated body part 331a may face a direction parallel to the direction in which the palm of the body 330 faces, and the other surface of the first designated body part 331a may face a direction parallel to the direction in which the back of the hand of the body 330 faces, but is not limited thereto. In case that the shapes of the plurality of markers 350 disposed on the one designated body part 331a are different from each other, the electronic device 310 according to an embodiment may quickly identify the angle of the first joint 332 based on identifying the shape of the marker 350. For example, in case that the shapes of the plurality of markers 350 disposed on the first designated body part 331a are not distinct from each other, the electronic device 310 may not be able to quickly obtain the angle of the first joint 332 because it needs to perform a complicated calculation process of obtaining a coordinate of another body part other than the first joint 332 to obtain the angle of the first joint 332. The electronic device 310 according to an embodiment may quickly identify the angle of the first joint 332 through the shape of the marker 350, thereby reducing the amount of calculation required to identify the first joint 332.

As described above, since the electronic device 310 according to an embodiment may identify the first joint 332 included in the distal part 331 of the body 330 based on the marker 350, it is possible to quickly reconstruct the posture of the body 330 and may reduce the amount of calculation required to identify the first joint 332.

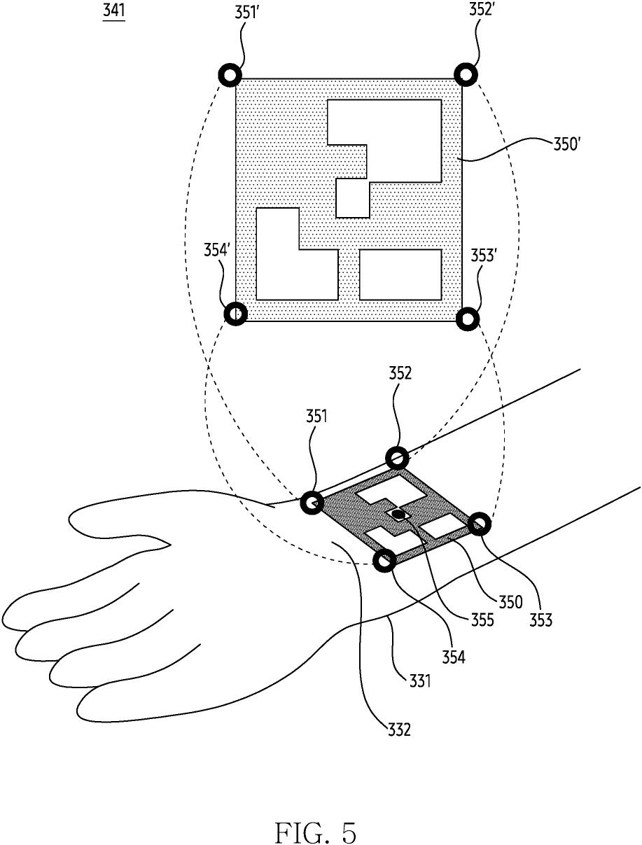

FIG. 5 illustrates an example of a method of identifying at least one first joint of a body based on a marker by an electronic device according to an embodiment.

Referring to FIG. 5, it is possible to know a method of identifying the first joint 332 based on a marker 350 included in a first region (e.g., a first region 341 of FIG. 4) by the electronic device (e.g., an electronic device 310 of FIG. 3) in more detail.

According to an embodiment, a processor of the electronic device 310 may obtain first information based on comparing the marker 350 captured in the first region 341 of a video 340 with a reference marker 350′ captured in another video pre-stored in a memory of the electronic device 310. The processor may identify different points 351, 352, 353, and 354 of the marker 350 in the first region 341. For example, the processor may identify the first point 351, the second point 352, the third point 353, and the fourth point 354, respectively, corresponding to the corners of the quadrangle marker 350. The processor may obtain coordinates of the different points 351, 352, 353, and 354 of the marker 350 based on comparing each of the different points 351, 352, 353, and 354 of the marker 350 with each of the different points 351′, 352′, 353′, and 354′ of the reference marker 350′ of the different video pre-stored in the memory. For example, the processor may obtain coordinates of the different points 351, 352, 353, and 354 of the marker 350 by comparing the first reference point 351′ of the reference marker 350′ corresponding to the first point 351 of the marker 350, the second reference point 352′ of the reference marker 350′ corresponding to the second point 352 of the marker 350, the third reference point 353′ of the reference marker 350′ corresponding to the third point 353 of the marker 350, and the fourth reference point 354′ of the reference marker 350′ corresponding to the fourth point 354 of the marker 350, respectively. The obtained coordinates of the different points 351, 352, 353, and 354 of the marker 350 may indicate the position of the different points 351, 352, 353, and 354 of the marker 350 based on a coordinate system defined in the first region 341. For example, the obtained coordinates of the different points 351, 352, 353, and 354 of the marker 350 may be expressed as a three-dimensional coordinate system, but is not limited thereto.

According to an embodiment, the processor of the electronic device 310 may identify the position of at least one first joint 332 based on obtaining the coordinates of the different points 351, 352, 353, and 354 of the marker 350. For example, the processor may obtain a first coordinate 355 indicating a center of the different points 351, 352, 353, and 354 of the marker 350. According to an embodiment, the processor may identify the first coordinate 355, which is the center of the different points 351, 352, 353, and 354 of the marker 350, as the coordinate of the first joint 332 of the body (e.g., a body 330 of FIG. 3).

According to an embodiment, the processor of the electronic device 310 may identify an angle of at least one first joint 332 based on the direction of the marker 350 indicated by the different points 351, 352, 353, and 354 of the marker 350. For example, the processor may obtain a normal vector of a plane defined by the different points 351, 352, 353, and 354 of the marker 350 in the first region 341, based on the coordinates of the different points 351, 352, 353, and 354 of the marker 350. The processor may identify the angle of the first joint 332 of the body 330 based on identifying the normal vector of the plane defined by the different points 351, 352, 353, and 354 of the marker 350.

According to an embodiment, the processor of the electronic device 310 may obtain the first information for indicating the position and angle of the first joint 332 based on the different points 351, 352, 353, and 354 of the marker 350. The first information may indicate the position and angle of the first joint 332 based on the coordinate system defined in the first region 341. For example, the first information may be expressed based on a three-dimensional coordinate system, but is not limited thereto.

As described above, the electronic device 310 according to an embodiment may quickly obtain first information for indicating the position and angle of the first joint 332 by comparing the different points 351, 352, 353, and 354 of the marker 350 with the different points 351′, 352′, 353′, and 354′ of the reference marker 350′ of another video pre-stored in the memory.

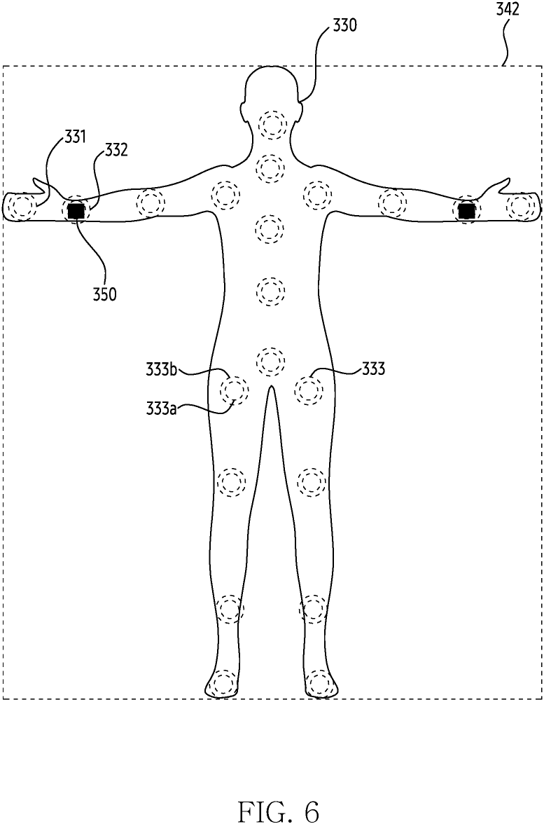

FIG. 6 illustrates an example of a method of identifying at least one second joint of a body based on a video by an electronic device according to an embodiment.

Referring to FIG. 6, it is possible to know a method of obtaining second information including the position of at least one second joint 333 based on a second region (e.g., a second region 342 of FIG. 4) by the electronic device (e.g., an electronic device 310 of FIG. 3) in more detail.

According to an embodiment, the processor of the electronic device 310 may obtain second information indicating a probability that at least one of at least one first joint 332 and at least one second joint 333 exists in a virtual two-dimensional space through the second model (e.g., a neural network 200 of FIG. 2) which received the second region 342. For example, the processor may obtain the second information indicating the probability that at least one first joint 332 and at least one second joint 333 exist in the virtual two-dimensional space through the second model. According to an embodiment, the second information may include the coordinate regarding the position of at least one first joint 332 and at least one second joint 333, based on a coordinate system defined in the second region 342.

According to an embodiment, the second information may indicate the probability that at least one first joint 332 and at least one second joint 333 exist in the form of a heat map. For example, in the second information, a region 333a may indicate a region having a relatively higher probability that the second joint 333 exists than a region 333b. In the second information, the processor may identify the position of the second joint 333 as a center of the region 333a. According to an embodiment, the processor may indicate the region 333a in which the probability of the second joint 333 existing is relatively high as a red series, and may indicate the region 333b in which the probability of the second joint 333 existing is relatively low as a blue series, but is not limited thereto.

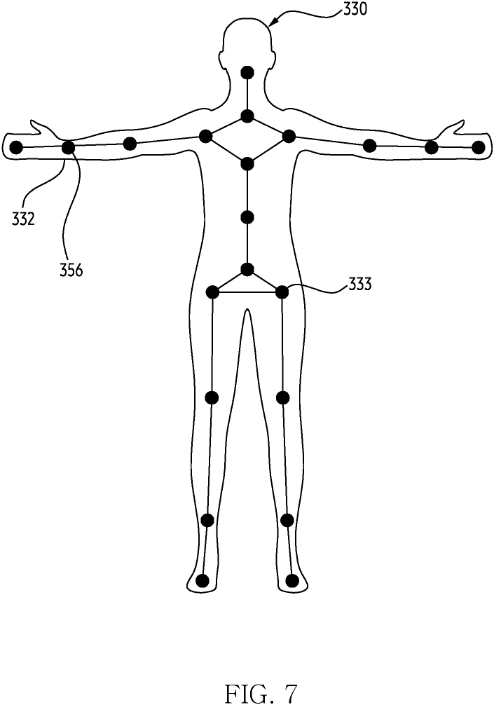

FIG. 7 illustrates an example of information indicating a posture of a body obtained by an electronic device according to an embodiment.

Referring to FIG. 7, it is possible to know a method of obtaining third information for indicating a posture of a body 330 by the electronic device (e.g., an electronic device 310 of FIG. 3) in more detail.

According to an embodiment, the processor of the electronic device 310 may obtain the third information indicating the posture of the body 330 based on the interconnection of at least one first joint 332 and at least one second joint 333, based on first information and second information. For example, the processor may obtain the third information by combining the position of at least one second joint 333 among the position of the first joint 332 included in the second information and the position of at least one second joint 333, with the position of at least one first joint 332 included in the first information. In other words, the processor may obtain the third information by discarding data on at least one first joint 332 included in the second information and combining other data on at least one second joint 333 included in the second information with another data on the position of the first joint 332 included in the first information. Since the accuracy of the position of the first joint 332 of the first information obtained based on the marker 350 is higher than the accuracy of the position of the first joint 332 of the second information obtained based on the second model, the electronic device 310 may reflect only the position of the first joint 332 included in the first information in the third information.

According to an embodiment, the processor of the electronic device 310, may convert a first coordinate (e.g., a first coordinate 355 of FIG. 4) into a second coordinate 356 defined in a second region (e.g., a second region 342 of FIG. 4), based on obtaining the first information, and may obtain the third information indicating the posture of the body 330 based on the second coordinate 356 and the second information. Since the first coordinate 355 obtained based on a first region (e.g., a first region 341 of FIG. 4) is a coordinate expressed based on the coordinate system defined in the first region 341, it may reflect only a relative position in the first region 341. The processor of the electronic device 310 may convert the first coordinate expressed based on the coordinate system defined in the first region 341 into the second coordinate based on the coordinate system defined in the second region 342. The second coordinates indicating the positions of one or more first joints 332 may be combined with the coordinate indicating the positions of one or more second joints 333 included in the second information.

According to an embodiment, the third information may indicate the posture of the body 330 by interconnecting the coordinate indicating the position of at least one first joint 332 and the coordinate indicating the position of at least one second joint 333. For example, the third information may be referred to as a skeleton model for indicating the posture of the body 330, but is not limited thereto. According to an embodiment, the third information may include data on an angle of at least one first joint 332. For example, since the first information includes data on the angle of at least one first joint 332 based on the marker 350, the third information obtained by combining the first information and the second information may include data on the angle of at least one first joint 332.

As described above, the electronic device 310 according to an embodiment may quickly and accurately reconstruct the posture of the body 330 by obtaining the first information indicating the position and angle of one or more first joints 332 based on the marker 350.

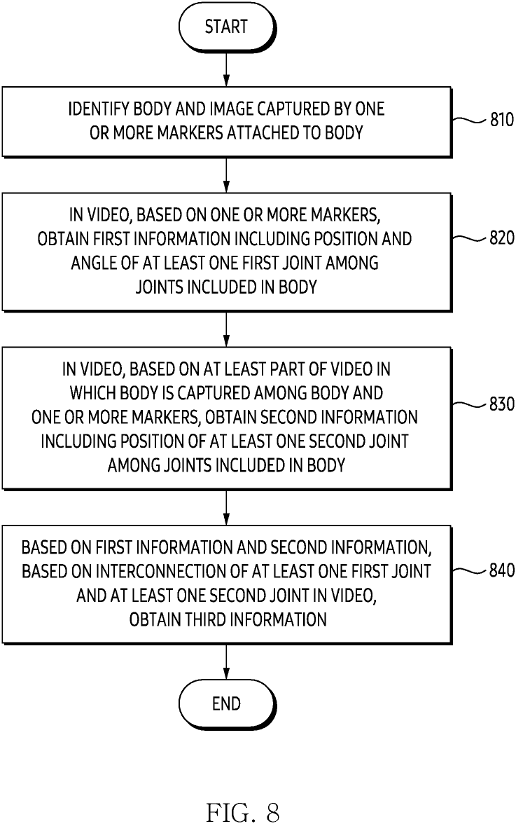

FIG. 8 is a flowchart for illustrating an operation of an electronic device according to an embodiment.

The operation illustrated in FIG. 8 may be performed by an electronic device 310 illustrated in FIG. 3.

Referring to FIG. 8, in operation 810, a processor of the electronic device 310 may identify a video (e.g., a video 340 of FIG. 3) in which a body (e.g., a body 330 of FIG. 3) and one or more markers (e.g., a marker 350 of FIG. 3) attached to the body 330 are captured. According to an embodiment, the marker 350 may be attached to at least a part of a distal part 331 so as to correspond to at least one first joint (e.g., at least one first joint 332 of FIG. 3) included in the distal part (e.g., the distal part 331 of FIG. 3). According to an embodiment, the processor may identify a first region (e.g., a first region 341 of FIG. 4) in the video 340 including one or more markers 350 and a body part corresponding to at least one first joint 332, and a second region (e.g., a second region 342 of FIG. 4) in the video 340 distinguished by the body 330, based on the first model which received the video 340. For example, the first region 341 may mean a partial region of the video 340 including the distal part (e.g., the distal part 331 of FIG. 3) of the body 330 including at least one first joint 332. For another example, the second region 342 may mean at least a partial region of the video 340 partitioned by the body 330.

In operation 820, the processor of the electronic device 310 may obtain first information including the position and angle of at least one first joint 332 among joints included in the body 330 based on one or more markers 350, in the video. According to an embodiment, the processor may obtain the first information, based on identifying different points (e.g., different points 351, 352, 353, and 354 of FIG. 5) of the marker 350, in the first region 341. For example, the processor may obtain the position of at least one first joint 332 based on identifying a first coordinate (e.g., a first coordinate 355 of FIG. 5) that is a center of the different points 351, 352, 353, and 354. For another example, the processor may identify the angle of at least one first joint 332 based on identifying a normal vector of a plane defined by the different points 351, 352, 353, and 354 in the first region 341. According to an embodiment, the first information may be in the form of three-dimensional coordinates expressed based on a coordinate system defined in the first region 341, but is not limited thereto.

In operation 830, the processor of the electronic device 310 may obtain second information including the position of at least one second joint 333 among joints included in the body 330 based on at least a part of the video 340 in which the body 330 is captured among the body 330 and one or more markers 350, in the video 340. According to an embodiment, the processor may obtain the second information indicating a probability that at least one of at least one first joint 332 and at least one second joint 333 exists in a virtual two-dimensional space, based on the second model which identified the second region 342. For example, the second information may be expressed as a heat map indicating the probability that at least one first joint 332 and at least one second joint 333 exist. According to an embodiment, the second information may be expressed based on the coordinate system defined in the second region 342.

In operation 840, the processor of the electronic device 310 may obtain third information indicating the posture of the body 330 based on the interconnection of at least one first joint 332 and at least one second joint 333 in the video 340, based on the first information and the second information. According to an embodiment, the processor may obtain the third information by combining the position of the at least one second joint 333 among the positions of at least one first joint 332 and the positions of the at least one second joint 333 included in the second information, with the positions of at least one first joint 332 included in the first information. In other words, the processor may obtain the third information by discarding data indicating the position of at least one first joint 332 included in the second information and combining another data indicating the position of at least one second joint 333 included in the second information with data indicating the position of at least one first joint 332 included in the first information.

According to an embodiment, the processor may be defined in the first region, and may convert the first coordinate 355 for indicating the position of at least one first joint 332 into a second coordinate defined in the second region 342. The processor may obtain the third information indicating the posture of the body 330 based on the converted second coordinate and the second information. According to an embodiment, the third information may indicate the posture of the body 330 by interconnecting the coordinate indicating the position of at least one first joint 332. For example, the third information may be referred to as a skeleton model for indicating the posture of the body 330.

As described above, the electronic device 310 according to an embodiment may quickly and accurately reconstruct the posture of the body 330 by obtaining the first information indicating the position and angle of one or more first joints 332 based on the marker 350.

An electronic device according to an embodiment (e.g., the electronic device 310 of FIG. 3) may comprise a memory for storing instructions, and at least one processor, wherein when the instructions are executed, the at least one processor may be configured to identify a video (e.g., a video 340 of FIG. 3) capturing a body (e.g., a body 330 of FIG. 3) and one or more markers (e.g., one or more markers 350 of FIG. 3) attached to the body, obtain a first information including an angle and a position of at least one first joint (e.g., at least one first joint 332 of FIG. 3) among joints being included in the body, based on the one or more markers in the video, obtain a second information including a position of at least one second joint (e.g., at least one second joint 333 of FIG. 3) among the joints being included in the body, based on at least part of the video in which the body is captured among the body and the one or more markers in the video, and obtain a third information indicating a posture of the body based on the interconnection of the at least one first joint and the at least one second joint in the video, based on the first information and the second information.

According to an embodiment, when the instructions are executed, the at least one processor may be further configured to identify a first region (e.g., a first region 341 of FIG. 4) in the video including the one or more markers and a body part corresponding to the at least one first joint, and a second region (e.g., the second region 342 of FIG. 4) in the video distinguished by the body, based on a first model receiving the video, obtain the first information including the angle and the position of at least one first joint, based on a plurality of coordinates indicating distinct points (e.g., distinct points 351, 352, 353, and 354 of FIG. 5) of the one or more markers in the first video, obtain the second information including the position of the at least one second joint, based on identifying the second region.

According to an embodiment, the plurality of coordinates respectively corresponds to each of the corners of a square marker of one or more markers, wherein when the instructions are executed, the processor may be further configured to obtain the first information, based on a center of the corners (e.g., a first coordinate 355 of FIG. 5) being identified by the plurality of coordinates.

According to an embodiment, the first information may include a first coordinate (e.g., a first coordinate 355 of FIG. 5) for indicating the position of at least one first joint, the first coordinate is defined in the first region, wherein when the instructions are executed, the at least one processor may be further configured to transform the first coordinate to a second coordinate (e.g., a second coordinate 356 of FIG. 7) defined in the second region, based on obtaining the first information, obtain the third information indicating the posture of the body, based on the second coordinate and the second information.

According to an embodiment, when the instructions are executed, the at least one processor may be further configured to obtain the angle of the at least one first joint based on a direction of the one or more markers being indicated by the plurality of coordinates.

According to an embodiment, when the instructions are executed, the at least one processor may be further configured to, through a second model receiving the second region, obtain the second information indicating the possibility that at least one of the first joint and at least one of the second joint exists in a virtual two-dimensional space.

According to an embodiment, when the instructions are executed, the at least one processor may be further configured to obtain the third information by combining the position of the at least one second joint, among a position of the at least one first joint being included in the second information and the position of the at least one second joint, and the position of the at least one first joint being included in the first information.

According to an embodiment, the memory may pre-store another video capturing each of the one or more markers before the video is captured, wherein when the instructions are executed, the at least one processor may be further configured to obtain the first information based on comparing each of the one or more markers being captured in the video and the one or more markers (e.g., a reference marker 350′ of FIG. 5) being captured in the other video and pre-stored in the memory.

According to an embodiment, the one or more markers respectively attached to a body part corresponding to a position of the at least one first joint.

According to an embodiment, the one or more markers may include a first marker (e.g., a first marker 350a of FIG. 4) attached to one surface of a body part corresponding to the one first joint; and a second marker (e.g., a second marker 350b of FIG. 4) attached to the other surface of the body part facing the one surface and corresponding to the one first joint, and the second marker has a shape distinct from the first marker.

According to an embodiment, the one or more markers may include a plurality of markers each disposed on a different body parts (e.g., body parts 331a and 331b of FIG. 4) of the body, a part of the plurality of markers may have a shape the same as each other.

According to an embodiment, an operating method of an electronic device (e.g., an electronic device 310 of FIG. 3) may comprise identifying a video (e.g., a video 340 of FIG. 3) capturing a body (e.g., a body 330 of FIG. 3) and one or more markers (e.g., one or more markers 350 of FIG. 3) attached to the body, obtaining a first information including an angle and a position of at least one first joint (e.g., at least one first joint 332 of FIG. 3) among joints being included in the body, based on the one or more markers in the video, obtaining a second information including a position of at least one second joint (e.g., at least one second joint 333 of FIG. 3) among the joints being included in the body, based on at least part of the video in which the body is captured among the body and the one or more markers in the video, and obtaining a third information indicating a posture of the body based on the interconnection of the at least one first joint and the at least one second joint in the video, based on the first information and the second information.

According to an embodiment, identifying the video may include identifying a first region (e.g., a first region of FIG. 4) in the video including the one or more markers and a body part corresponding to the at least one first joint, and a second region (e.g., a second region 342 of FIG. 4) in the video distinguished by the body, based on a first model receiving the video, wherein obtaining the first information may include obtaining the first information including the angle and the position of at least one first joint, based on a plurality of coordinates indicating distinct points (e.g., distinct points 351, 352, 353, and 354 of FIG. 5) of the one or more markers in the first video, and wherein obtaining the second information may include obtaining the second information including the position of the at least one second joint based on identifying the second region.

According to an embodiment, the first information may include a first coordinate (e.g., a first coordinate 355 of FIG. 5) for indicating the position of at least one first joint, the first coordinate is defined in the first region, and wherein obtaining the third information may include transforming the first coordinate to a second coordinate (e.g., a second coordinate 356 of FIG. 7) defined in the second region, based on obtaining the first information, and obtaining the third information indicating the posture of the body, based on the second coordinate and the second information.

According to an embodiment, obtaining the first information may further include obtaining the angle of the at least one first joint based on a direction of the one or more markers being indicated by the plurality of coordinates.

According to an embodiment, obtaining the second information may include obtaining the second information indicating the possibility that at least one of at least one the first joint and at least one the second joint exists in virtual two-dimensional space, through a second model receiving the second region.

According to an embodiment, obtaining the third information may include obtaining the third information by combining the position of the at least one second joint, among a position of the at least one first joint being included in the second information and the position of the at least one second joint, and the position of the at least one first joint being included in the first information.

According to an embodiment, the one or more markers respectively may be attached to a body part corresponding to a position of the at least one first joint.

According to an embodiment, a computer readable storage medium storing one or more programs, when executed by at least one processor of electronic device (e.g., an electronic device 310 of FIG. 3), the one or more programs may cause the electronic device to obtain a first region (e.g., a 1-1 region 341a of FIG. 4) capturing a marker (e.g., a marker 350 of FIG. 3) attached to a body and a second region (e.g., a second region 342 of FIG. 4) capturing the body and at least partially overlapped to the first region, from a video (e.g., a video 340 of FIG. 3) capturing the body (e.g., a body 330 of FIG. 3), identify a position of a designated body part (e.g., at least one first joint 332 of FIG. 3) among a plurality of body parts of the body, based on the marker being included in the first region, identify a position of the plurality of body parts (e.g., at least one second joint 333 of FIG. 3) being included in the body in the second region, from the second region, obtaining an information indicating a posture of the body, based on the position of the plurality of body parts being identified in the second region and the position of the designated body part being identified based on the marker.

According to an embodiment, when executed by at least one processor of an electronic device, the one or more programs further may cause the electronic device to obtain a third region (e.g., a 1-2 region 341b of FIG. 4) capturing a second marker distinct from the first marker which is the marker, from the video, and identify a position of a second designated body part (e.g., a first designated body part 331b of FIG. 4) corresponding to the first designated body part (e.g., a first designated body part 331a of FIG. 4) which is the designated body part in the body, based on the second marker being included in the third region.

The electronic device according to various embodiments disclosed in the present document may be a device of various type. The electronic device may include, for example, a portable communication device (e.g., a smartphone), a computer device, a portable multimedia device, a portable medical device, a camera, a wearable device, a server, or a home appliance. The electronic device according to an embodiment of the present document is not limited to the above-described devices.

The various embodiments and terms used in the present document are not intended to limit the technical features described herein to specific embodiments and should be understood to include various modifications, equivalents, or substitutes of the embodiment. With respect to the description of the drawings, similar reference numerals may be used for similar or related components. The singular form of the noun corresponding to the item may include one or more of the items unless clearly indicated differently in a related context. In the present document, each of the phrases such as “A or B”, “at least one of A and B”, “at least one of A or B,”, “A, B or C,”, “at least one of A, B and C”, and “at least one of A, B, or C” may include any one of the phrases together, or all possible combinations thereof. Terms such as “1st”, “2nd”, or “first”, or “second” may be used simply to distinguish a corresponding component from another corresponding component, and are not limited to other aspects (e.g., importance or order). When some (e.g., the first) component is referred to as “coupled” or “connected” in another (e.g., the second) component, with or without the term “functional” or “communicatively”, it means that some of the components may be connected directly (e.g., wired), wirelessly, or through a third component.

The term “module” used in the present document may include a unit implemented in hardware, software, or firmware and be used interchangeably with terms such as logic, logic block, component, or circuit, and the like, for example. The module may be a minimum unit or a part of the integrally configured component or the component that performs one or more functions. For example, according to an embodiment, the module may be implemented in the form of an application-specific integrated circuit (ASIC).

Various embodiments of the present document may be implemented as software (e.g., a program) including one or more instructions stored in a storage medium (or external memory) readable by a device (e.g., wearable device 100). For example, a processor (e.g., a processor) of a device (e.g., wearable device 100) may call and execute at least one of the one or more instructions stored from a storage medium. This makes it possible for the device to operate to perform at least one function according to the at least one command called. The one or more instructions may include code generated by a compiler or code that may be executed by an interpreter. The device-readable storage medium may be provided in the form of a non-transitory storage medium. Here, the term ‘non-transitory’ only means that a storage medium is a device that is tangible and does not include a signal (e.g., electromagnetic wave), and the term does not distinguish between a case where data is semi-permanently stored and a case where it is temporarily stored.

According to an embodiment, a method according to various embodiments disclosed in the present document may be provided by being included in a computer program product. The computer program products may be traded between sellers and buyers as products. The computer program products may be distributed in the form of device-readable storage media (e.g., compact disc read only memory (CD-ROM), or distributed (e.g., downloaded or uploaded) directly or online through an application store (e.g., Play Store™, or App Store™) or between two user devices (e.g., smartphones). In the case of online distribution, at least some of the computer program products may be temporarily stored or temporarily created on a device-readable storage medium such as a manufacturer’s server, a server in an application store, or a memory in a relay server.

According to various embodiments, each of the above-described components (e.g., a module or a program) may include a single object or a plurality of objects. According to various embodiments, one or more components or operations of the above-described corresponding components may be omitted, or one or more other components or operations may be added. Alternatively, or additionally, a plurality of components (e.g., modules or programs) may be integrated into one component. In this case, the integrated component may perform one or more functions of component of each of the plurality of components in the same or similar manner as those performed by the corresponding component among the plurality of components before the integration. According to various embodiments, operations performed by a module, a program, or other components may be executed sequentially, in parallel, repeatedly, or heuristically, performed, one or more of the operations are executed in a different order, omitted, or one or more other operations may be added.

What is claimed is:

1. An electronic device comprising:
A memory for storing instructions; and
At least one processor;
wherein when the instructions are executed, the at least one processor is configured to:
identify a video capturing a body and one or more markers attached to the body,
obtain a first information including an angle and a position of at least one first joint among joints being included in the body, based on the one or more markers in the video,
obtain a second information including a position of at least one second joint among joints being included in the body, based on at least part of the video in which the body is captured among the body and the one or more markers in the video, and
obtain a third information indicating a posture of the body based on the interconnection of the at least one first joint and the at least one second joint in the video, based on the first information and the second information.

2. The electronic device of claim 1,
wherein when the instructions are executed, the at least one processor is further configured to:
identify a first region in the video including the one or more markers and a body part corresponding to the at least one first joint, and a second region in the video distinguished by the body, based on a first model receiving the video,
obtain the first information including the angle and the position of at least one first joint, based on a plurality of coordinates indicating distinct points of the one or more markers in the first video,
obtain the second information including the position of the at least one second joint, based on identifying the second region.

3. The electronic device of claim 2,
wherein the plurality of coordinates respectively corresponds to each of the corners of a square marker of one or more markers,
wherein when the instructions are executed, the at least one processor is further configured to:
obtain the first information, based on a center of the corners being identified by the plurality of coordinates.

4. The electronic device of claim 2,
wherein the first information includes a first coordinate for indicating the position of at least one first joint, the first coordinate is defined in the first region,
wherein when the instructions are executed, the at least one processor is further configured to:
transform the first coordinate to a second coordinate defined in the second region, based on obtaining the first information,
obtain the third information indicating the posture of the body, based on the second coordinate and the second information.

5. The electronic device of claim 2,
wherein when the instructions are executed, the at least one processor is further configured to:
obtain the angle of the at least one first joint based on a direction of the one or more markers being indicated by the plurality of coordinates.

6. The electronic device of claim 2,
wherein when the instructions are executed, the at least one processor is further configured to:
through a second model receiving the second region, obtain the second information indicating the possibility that at least one of the first joint and at least one of the second joint exists in a virtual two-dimensional space.

7. The electronic device of claim 6,
wherein when the instructions are executed, the at least one processor is further configured to:
obtain the third information by combining the position of the at least one second joint, among a position of the at least one first joint being included in the second information and the position of the at least one second joint, and the position of the at least one first joint being included in the first information.

8. The electronic device of claim 1,
wherein the memory pre-stores another video capturing each of the one or more markers before the video is captured,
wherein when the instructions are executed, the at least one processor is further configured to:
obtain the first information based on comparing each of the one or more markers being captured in the video and the one or more markers being captured in the other video and pre-stored in the memory.

9. The electronic device of claim 1,
wherein the one or more markers respectively attached to a body part corresponding to a position of the at least one first joint.

10. The electronic device of claim 1,
wherein the one or more markers includes:
a first marker attached to one surface of a body part corresponding to the at least one first joint; and
a second marker attached to the other surface of the body part facing the one surface and corresponding to the at least one first joint, and the second marker has a shape different from the first marker.

11. The electronic device of claim 1,
wherein the one or more markers includes a plurality of markers each disposed on a different body part of the body,
wherein a part of the plurality of markers has a shape the same as each other.

12. An operating method of an electronic device comprising:
identifying a video capturing a body and one or more markers attached to the body,
obtaining a first information including an angle and a position of at least one first joint among joints being included in the body, based on the one or more markers in the video,
obtaining a second information including a position of at least one second joint among the joints being included in the body, based on at least part of the video in which the body is captured among the body and the one or more markers in the video, and
obtaining a third information indicating a posture of the body based on the interconnection of the at least one first joint and the at least one second joint in the video, based on the first information and the second information.

13. The method of claim 12,
wherein identifying the video includes identifying a first region in the video including the one or more markers and a body part corresponding to the at least one first joint, and a second region in the video distinguished by the body, based on a first model receiving the video,
wherein obtaining the first information includes obtaining the first information including the angle and the position of at least one first joint, based on a plurality of coordinates indicating distinct points of the one or more markers in the first video, and
wherein obtaining the second information includes obtaining the second information including the position of the at least one second joint based on identifying the second region.

14. The method of claim 13,
wherein the first information includes a first coordinate for indicating the position of at least one first joint, the first coordinate is defined in the first region, and
wherein obtaining the third information includes:
transforming the first coordinate to a second coordinate defined in the second region, based on obtaining the first information, and
obtaining the third information indicating the posture of the body, based on the second coordinate and the second information.

15. The method of claim 13,
wherein obtaining the first information further includes obtaining the angle of the at least one first joint based on a direction of the one or more markers being indicated by the plurality of coordinates.

16. The method of claim 13,
wherein obtaining the second information includes obtaining the second information indicating the possibility that at least one of at least one the first joint and at least one the second joint exists in virtual two-dimensional space, through a second model receiving the second region.

17. The method of claim 16,
wherein obtaining the third information includes obtaining the third information by combining the position of the at least one second joint, among a position of the at least one first joint being included in the second information and the position of the at least one second joint, and the position of the at least one first joint being included in the first information.

18. The method of claim 12,
wherein the one or more markers respectively attached to a body part corresponding to a position of the at least one first joint.

19. A computer readable storage medium storing one or more programs, when executed by at least one processor of an electronic device, the one or more program causes electronic device to:
obtain a first region capturing a marker attached to a body and second region capturing the body and at least partially overlapped to the first region, from a video capturing the body,
identify a position of a designated body part among a plurality of body parts of body, based on the marker being included in the first region,
identify a position of the plurality of body parts being included in the body in the second region, from the second region,
obtaining an information indicating a posture of the body, based on the position of the plurality of body parts being identified in the second region and the position of the designated body part being identified based on the marker.

20. The computer readable storage medium of claim 19,
wherein the marker is a first marker,
wherein the designated body part is a first designated body part, and
wherein when executed by at least one processor, the one or more program further causes electronic device to:
obtain a third region capturing a second marker distinct from the first marker from the video, and
identify a position of a second designated body part corresponding to the first designat ed body part, based on the second marker being included in the third region.
